(19) United States
(12) Patent Application Publication
(10) Pub. No.: US 2022/0285018 A1
(43) Pub. Date: Sep. 8, 2022

(54) System and Method for In-Person Encounters and Assistance for Remote or Noncorporeal Medical Diagnosis and Treatment

(71) Applicant: Joseph Alan Epstein, Pleasanton, CA (US)

(72) Inventor: Joseph Alan Epstein, Pleasanton, CA (US)

(21) Appl. No.: 17/750,366

(22) Filed: May 22, 2022

Related U.S. Application Data
(63) Continuation of application No. 16/826,273, filed on Mar. 22, 2020.

(51) Int. Cl. G16H 40/20; G16H 10/60; G16H 70/20; G16H 80/00; G06Q 30/04

(52) U.S. Cl. CPC G16H 40/20; G16H 10/60; G16H 70/20; G16H 80/00; G06Q 30/04; A61B 5/0053

(57) ABSTRACT

A method and system providing medical treatment to patients. In some embodiments, a remote practitioner is connected via a referral network to an in-person clinician that can perform work that cannot be performed remotely on behalf of the practitioner. Some embodiments perform a lightweight referral for said work, where the work may be smaller than the minimum procedure code and assigned billing for the overall specific therapy being undertaken. In some embodiments, the in-person clinician is only licensed to be able to perform the tasks they are assigned. In some embodiments, the in-person clinicians operate as the remote in-person medical assistance needed for the remote practitioner to practice medicine. Billing and pricing methods are disclosed for sub-procedure-code tasks.

### CROSS REFERENCE TO RELATED APPLICATIONS

This is a continuation of application Ser. No. 16/826,273, filed Mar. 22, 2020 by the present inventor, which claims the benefit of provisional patent application Ser. No. 62/822,829, filed Mar. 23, 2019 by the present inventor. All the foregoing applications are hereby incorporated herein by reference in their entirety.

### BACKGROUND OF THE INVENTION

### 1. Field of the Invention

The present invention relates to the field of the remote practice of medicine on patients, including integration with electronic health records, in-person clinics, and billing systems.

### 2. Description of the Prior Art

Telemedicine and remote medicine (online, phone, video chat, and so on) are new ways to deliver medical treatment to patients, brought with the promise of lower costs and easier use. However, except for a small number of diseases (such as rashes), it is impossible to properly detect diagnostic signs of most disorders. The standards of diagnosis may require palpation, auscultation, or direct measurement of the body and its reactions.

This stymies the goal of remote medicine, which is to be able to carry out the diagnosis and treatment remotely, thus allowing physicians to provide treatment virtually in far more places than they can be in physically.

One attempt at a solution is for the teledoc to refer the patient to be seen in an urgent care clinic. However, urgent care clinics are only set up to do the complete evaluation and treatment—often under a different copay scheme for the patient—and the current practice of assigning procedure codes to procedures tied to taxpayer identifiers and contracted billing rates interferes with being able to refer for minor subprocedures that fall within a total procedure code. Therefore, the systemic restrictions on billing for the referral force telemedinice today to act as mostly a front-line call center for urgent care clinics: problems that really only require verbal reassurance can be handled remotely, whereas other problems must escalate to the clinics. If there was an ongoing relationship of care between the remote practitioner and patient, the referral breaks it. Continuity of care is disrupted, and more expensive (such as urgent care) resources are locked in to handle the remainder of the case. This happens even if all the patient needed was a minimal visual inspection, for example—one that could have been handled by minimally licensed or unlicensed assistance.

Today's referral system is designed to identify one or more licensed practitioners for at least the work of one complete CPT code to be performed. There is no uniform way to refer out the work that an assistant or nurse would do, when that is a fraction of an entire encounter and is expected to be a part of the overall encounter code the remote practitioner hoped to bill for. This is a primary reason why most medical systems (hospitals and practices) today offer teledocs but are unable to bill for the effort under the principal procedure codes. All referrals that are made are coarse-grained, must by definition encompass one full procedure, and will be fully reimbursed only to the provider performing the procedure. For example, there is no way for a practitioner to take advantage of a retail clinic (such as a CVS MinuteClinic) as if the clinic were an extension or arm of the practitioner, such as full integration into the work that the practitioner should be doing and proper accounting and reimbursements or revenue sharing between the clinician and the practitioner to provide a seamless experience. This problem is because the CPT code must be performed by one billing center (TID)—CPTs are not fractionally billed. The traditional referral system is very heavyweight for what should be a lightweight problem of dispatch and retrieve.

Consider a primary care physician performing telemedicine, and he says that the patient should come in to get a knee flexed to determine the possibility of injury. The remote physician wants to create a referral, perhaps in this case to another PCP. But to whom? His electronic health record (EHR) does not offer the ability to filter down to only those practitioners currently in the office and ready to receive a patient. EHRs and schedulers are usually not integrated in practice or in construction, so he has no idea which in-person clinician might be available and when. Medical assistants and nurse practitioners might not even be available in the system as practitioners for such a referral, and so if the primary wants to send the patient to a more inexpensive or available resource, he cannot. Doing the referral may also lead to internal problems: a PCP referring to another PCP is unusual practice and may raise alarms within the practice and within the insurance company. The referral will also likely share diagnostic codes and CPT codes, or include CPT codes that are expected to be a part of the work of another CPT code, and thus may lead to reduced payment or rejection, especially because insurers often require the CPT code for the referred examination be suffixed (such as 25 for same day service of two different, unrelated encounters), or else their system will reject payment for the second as a double-billing error or an unauthorized second opinion. And that's if the electronic referral can even be made. Referrals in EHRs usually do not create a meaningful entry for scheduled work in another clinic's EHR. Instead, the patient is expected to call the second clinic, who can then look into their EHR—if it is connected to the primary's—to see the referral letter electronically. This may be appropriate for, say, a specialist referral, urgent or emergency care, or specialized testing. But that is not a seamless referral for a minor procedure.

Beyond that, the EHRs of today are not designed to handle minor matterst such as lightweight “referrals”. Imagine if in every PCP visit, which today always requires a medical assistant to perform intake and record impressions in advance of the PCP entering the room, a referral were generated to the medical assistant for that intake. This would mean that there would now be twice the number of encounters recorded in the EHR. Do both encounters get assigned billing codes? If so, what should the insurer pay? If not, how does internal audit separate this case from the case of encounters that ought to have been billed being skipped? And because there's no mechanism to true up any costs, do insurers get twice the bill entries and have twice the load on their systems with twice the work to do? Rejections may abound, or the insurer may pay an unfair distribution of reimbursements.

If the referral were to be recorded between two clinics, most insurers are likely to make the mistake of assigning the bulk of the reimbursement to the in-person clinician, even though the bulk should be given to the primary who is performing the medical diagnosis and taking the malpractice risk by being the provider of record. Even if the insurer could accept this, they might penalize the providers for overtaxing their system with two different providers creating reimbursement invoices for the same work that everyone else provides in only one invoice to one practitioner/TID. There needs to be a way for making these referrals outside of the EHR, or at least outside of the procedure code, unless the entire industry be forced to adopt a slew of new, trivial, usually uncounted microprocedures such as recordkeeping. And finally, even if the in-person clinician could do all this in the current system as mentioned above with all the tweaks and major changes, they would have a strong incentive to want to take over the patient relationship, because of the way insurers work (including any accountable care relationships or capitations), and because they—not having joined into an agreement with the referrer most likely—do not know that further revenue is available to them by taking the next referral and would for economic reasons try to retain the bird in the hand, thus defeating the purpose of the referrer.

### SUMMARY

In accordance with some embodiments, a method and system for delivering medical treatment to patients, thus causing their conditions to be altered in physically manifested ways, by allowing a remote practitioner to evaluate a patient and refer possibly small, sub-CPT procedures to an actual in-person clinician to perform. Tasks are sent along with the referral, and once performed, are entered into an EHR interface for updating the patient's chart so that the referring remote practitioner can see the results and administer treatment. In some embodiments, the tasks encompass tests, evaluations, and administration of therapies.

### BRIEF DESCRIPTION OF THE DRAWINGS

FIG. 1 is a diagram of an embodiment of the invention, illustrating the interaction of a remote practitioner through a referrer to an in-person clinician, across different EHR interfaces.

FIG. 2 is a diagram of an embodiment of the invention, further illustrating the involvement of a referred clinic.

FIG. 3 is a diagram of an embodiment of the invention, illustrating a bridge between separate EHR systems.

FIG. 4 is a diagram of an embodiment of the invention, showing scheduling and billing.

FIG. 5 is a diagram of an embodiment of the invention, showing common chart identification and access controls from the remote EHR interface.

FIG. 6 is a diagram of an embodiment of the invention, showing the clinician pricing and billing through a point of sale.

FIG. 7 is a diagram of an embodiment of the invention, showing collaboration between the patient and a referred clinician.

FIG. 8 is a diagram of an embodiment of the invention, showing a patient interacting with a robodoc and triggering referral to an in-person clinician.

### DETAILED DESCRIPTION

First disclosed are embodiments for a lightweight task (such as impression taking, procedure performing, or treatment providing) referral system, which can be operated independently from the traditional heavyweight EHR and procedure code driven model.

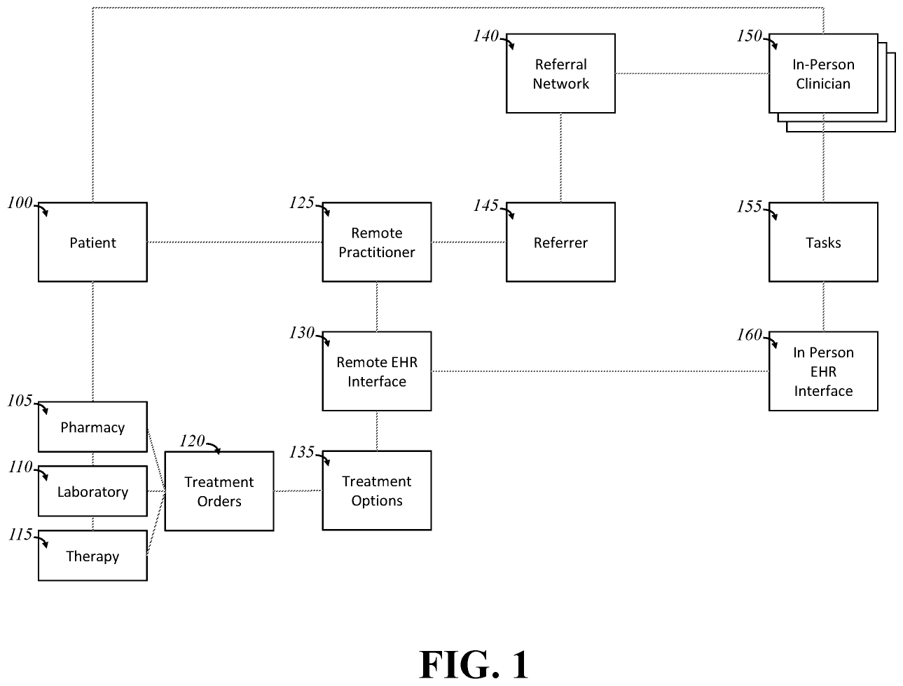

FIG. 1 shows the general architecture of some embodiments. A patient 100 and remote practitioner 125 interact. When the practitioner 125 requires a referral out to an in-person clinician 150, she requests the referral from a referrer 145. The referrer 145 accesses a referral network 140 to identify one or more in-person clinicians 150 who can perform the requested task or tasks 155. In some embodiments, the referrer searches the list and chooses or down-selects a list of candidate clinicians on the basis of, in whole or in part, one or more of the following: the cost of the clinician, the clinician's ability to perform the task, the clinician's skill or licensure or certification or quality measures taken about their ability to perform the task, the practitioner's preference, the patient's preference, distance from the patient, time the patient would wait, time the task would take, the time the results would be made available to the practitioner, the reputation of the clinician, other medical factors, other practical factors, and other economic factors. Once the referrer provides one or more options for the patient and practitioner, the patient sends herself or is sent to one of the in-person clinicians, where the task is performed, and the completion, along with whatever impressions are generated, are input into the in-person clinician's electronic health record interface 160. In some embodiments, the task 155 includes procedures. In some embodiments, the task includes treatments or therapies. In some embodiments, the task is marked up or presented with instructions on how to do the task. In some embodiments, the task is marked up with educational or step-by-step material for the clinician who will perform the task: in some further embodiments, the clinicians 150 need not be trained in advance or know the name of the procedure to perform, but instead are informed through the task dispatch on how to perform that task. The practitioner 125 will get the confirmation and/or results such as from the task via remote EHR interface 130 and, when warranted, can provide treatment orders 120 and cause treatments such as therapy 115, laboratory work from a laboratory 110, or prescriptions issued from a pharmacy 105 to be given to the patient and change his medical state further, beyond that which the task 155 has already performed. In some embodiments, the lightweight nature of the referral is maintained within the EHR system by reconciling the treatment orders and procedure codes thus booked with the work performed within the tasks 155 of the in-person clinician.

In some embodiments, remoteness is not a property of the Remote Practitioner 125. In fact, that practitioner can be local. Some embodiments use this referral system to refer out to a lower-licensed or less loaded resource, such as when the primary practitioner does not have time or the inclination to perform those parts of the evaluation. Some embodiments use the referrals to find clinicians who have a higher license or skill level, such as where the particular task to be performed is beyond the scope of the skill of the primary practitioner or is too complicated or involved for the practitioner to want to attempt it. The figure still applies, but substitute “Primary” for “Remote”.

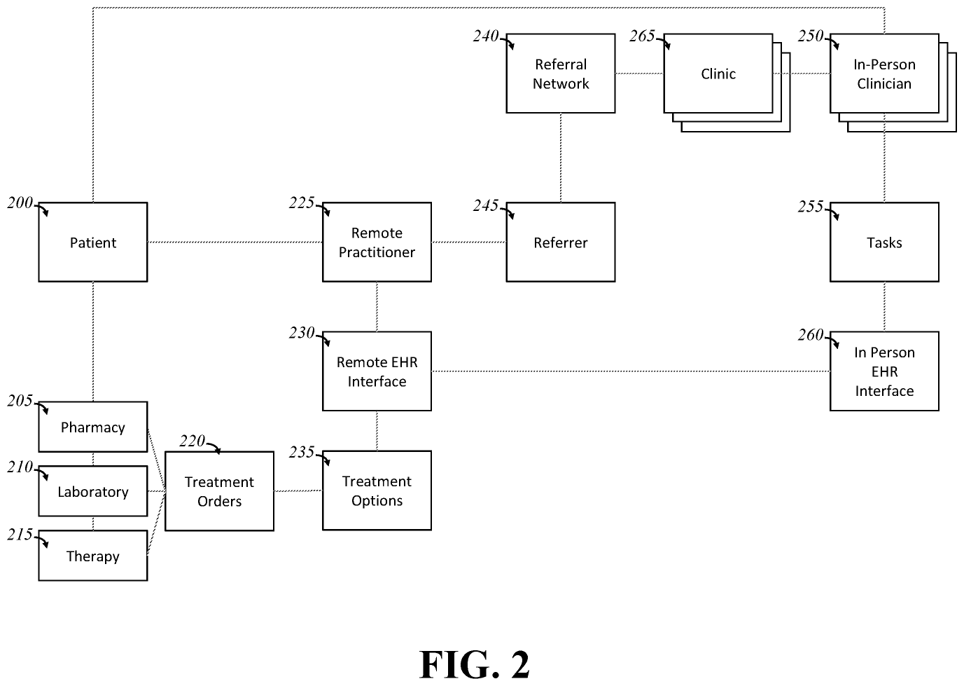

In some embodiments, some clinicians work together in shared clinics. FIG. 2 shows some embodiments that have, in the referral network 240, these clinics 265 with possibly multiple clinicians 250 each. In some of these embodiments, the referrals from referrer 245 are to clinics 265 and not clinicians 250 directly, and the clinic 265 will identify a clinician 250 to perform the task or tasks 255.

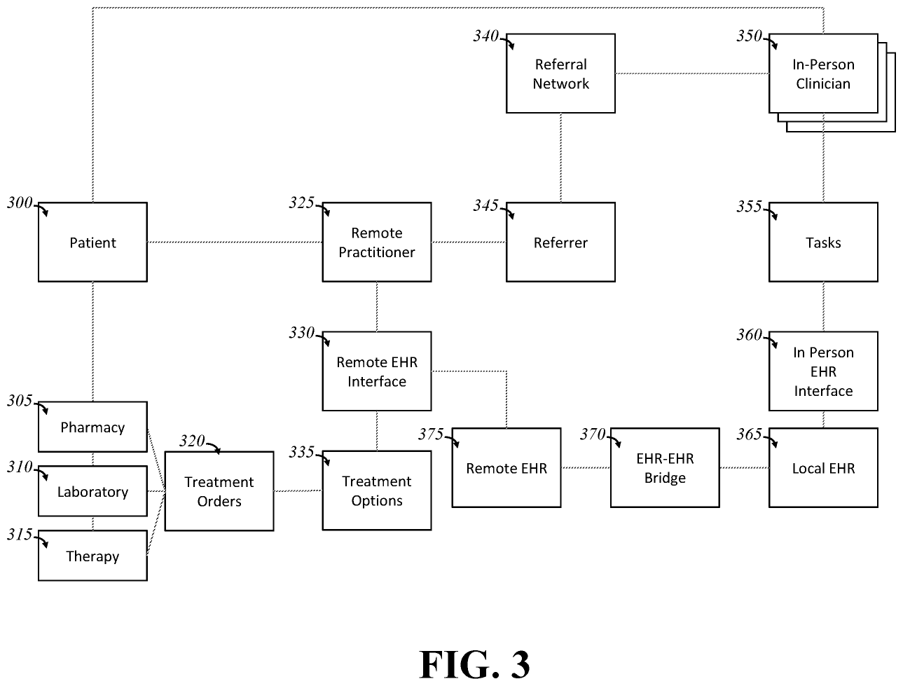

In some embodiments, the multiple EHR interfaces belong to the same EHR system. In some embodiments, such as shown in FIG. 3, the clinician EHR 365 is a different instance than the one the remote practitioner has 375, and therefore an EHR-to-EHR bridge 370 exchanges the information between the two systems regarding the patient.

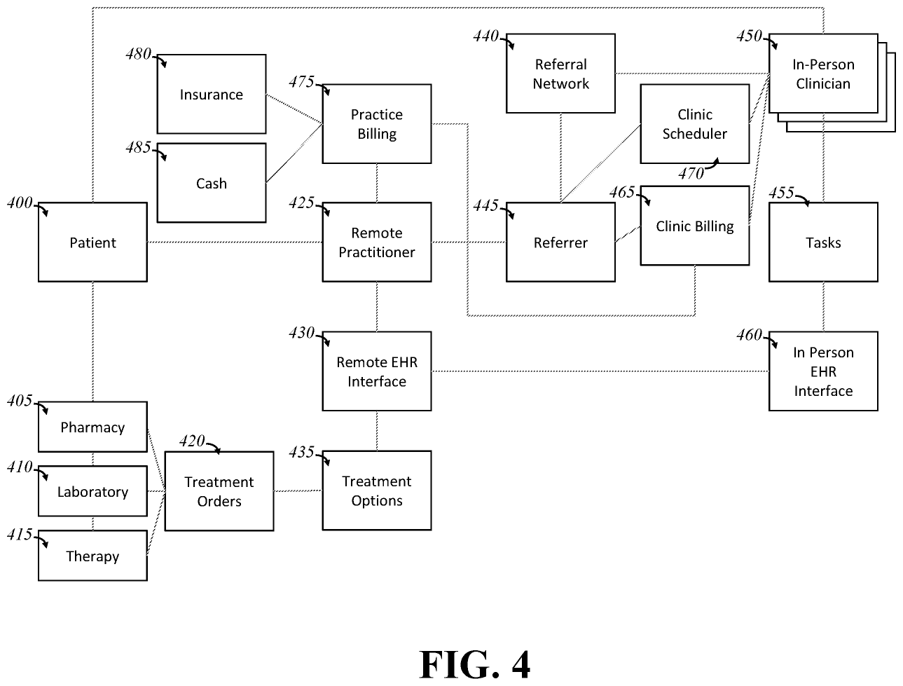

In some embodiments, such as illustrated in FIG. 4, some clinics maintain a clinic scheduling system. In some embodiments a referrer 445 accesses a clinic scheduler 470 to determine availability of a clinician or resource. In some embodiments the referrer 445 accesses the clinic scheduler 470 to create a schedule entry for the patient 400. In some further embodiments, the referrer 445 hands over sufficient information, such as the patient's Social Security Number or regional, universal, or clinic EHR local medical record number, plus whatever information that may need to be programmed into the EHR, to allow the clinician 450 to be able to access and update the patient chart via 460. In some embodiments the access and update allowed is complete. In some embodiments, access restrictions are applied. In some embodiments, the impressions and/or results are transmitted via a one-way channel: in some further embodiments, the information passed to the clinician includes the remote practitioner's EHR's medical record number (MRN) or similar identifier, plaintext or encrypted (some embodiment employ a one-way hash, such as SHA256 to identifier) to allow the clinician to provide data to the practitioner's EHR.

In some embodiments, some of the in-person clinicians are more inexpensive or plentiful than MDs, such as nurse practitioners or even medical assistants. In some embodiments, some of the in-person clinicians are physicians and doctors who take these referrals for task as a service. In some embodiments, the clinicians are practitioners at retail establishments (such as a CVS MinuteClinic). In some embodiments, the clinicians are mobile: in some further embodiments, the scheduler for the clinic determines the mobile clinician's route and workload. In some embodiments, the clinicians are “gig” workers, and the scheduler ensures that a clinician is available and willing, and handles cases of failover if a clinician cannot fulfill the request. In some embodiments, the patient is provided an application to see the status of the arrival time, location, and availability of the clinician. In some embodiments, the expected price is also displayed.

In some embodiments, the clinicians or their clinics bill the patient or collect the patient's insurance information and submit a claim and collect any due-at-service copays and coinsurances directly for the procedures that have been requested. In some embodiments, the remote billing system performs the billing of the patient or the insurance and provides a revenue share or service fee to the clinic billing system, thus satisfying the reimbursement without requiring the patient to be bothered. In some embodiments, the workflow is constructed so that the patient need merely identify herself to the clinician sufficiently to allow the clinician to access the records, be sure the person presenting is the patient, and to perform the task, with no further electronic or paper transactions needed from the patient: in some further embodiments, this information is transmitted using the patient's smartphone (such as with an app tied to this particular service). In some embodiments, the clinician's billing and the practitioner's are coordinated to ensure that insurance will cover both encounters (even though they logically could be said to be one encounter). In some embodiments, the clinician's and practitioner's billing systems are coordinated to ensure that only one of them performs the bill, and that all reconciliation and truing up occurs as needed per agreements. In some embodiments, the referral network is tagged with reimbursement agreements: in some, the referrer makes make proper determinations of the likelihood of additional complexity to the patient; in some, the clinician makes the proper determination of its desire or ability to take the patient and whether it can expect to get paid appropriately. In some embodiments, the clinician provides variable pricing: in some further embodiments, the clinician provides time of use discounts (such as for off hours); in some embodiments, the clinician provides affiliation discounts; in some embodiments, the clinician provides volume discounts. In some embodiments the discounts are reflected in the price shown to the selector (patient or practitioner). In some embodiments the discounts are not shown, to prevent violations of rules for kickbacks of referrals when both are billed. In some embodiments, the discounts accrue to the practitioner and not the patient.

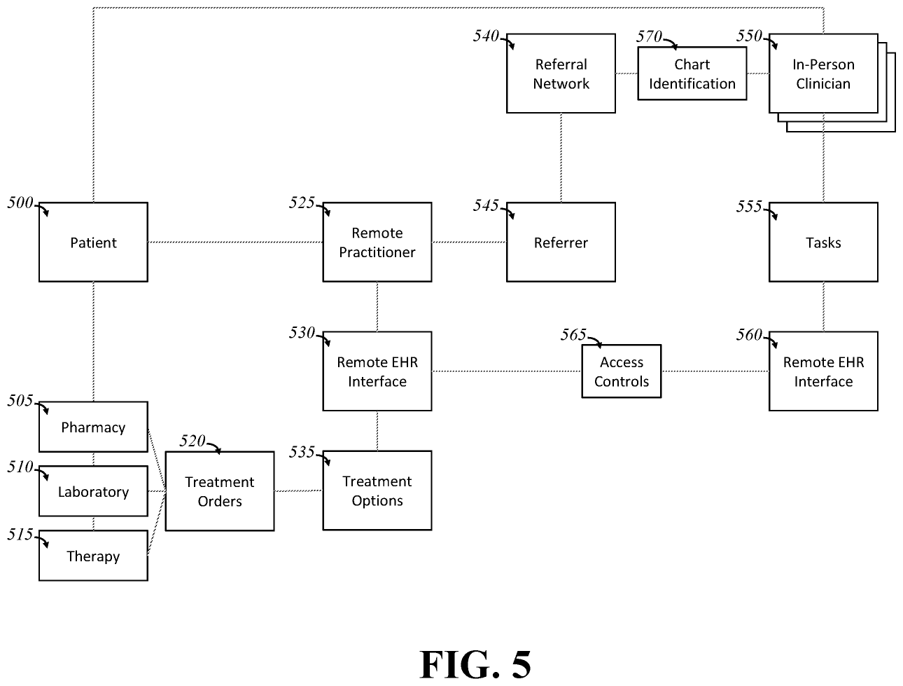

FIG. 5 shows some embodiments that provide such access restriction. A clinician 550 accesses a remote EHR interface 560, but with optional access controls 565. In some embodiments, the access controls limit the clinician's interface to post only, as in the impressions are sent in but nothing besides confirmations can be sent back: in some embodiments this is directly by using paper and facsimile, but the more likely embodiments to be employed are when the remote EHR interface is an application or website and the access controls are implemented electronically. In some embodiments, the access controls exist perforce via a reduced clinician portal specifically designed to control the flow of information to the clinic, in which case such portal is an embodiment of the remote EHR interface. In some embodiments the patient's social security number or medical record number is not transmitted in the clear: instead, the chart identification block 570 performs whatever translations or obfuscations are requested or necessary by configuration (such as to perform a one-way hash of the identification, as disclosed above). In some embodiments, the translated identifier is transmitted to the Remote EHR to reidentify the patient in the EHR system when the clinician provides new information.

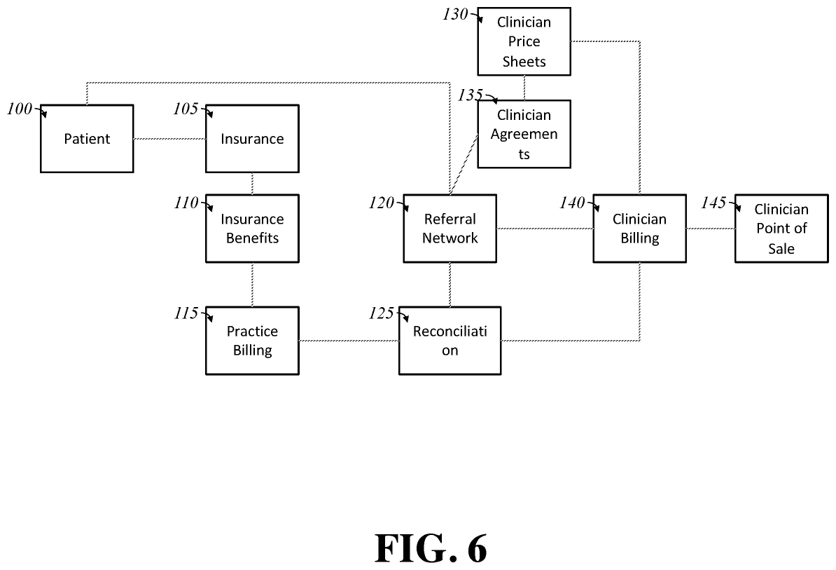

FIG. 6 shows the detailed clinic agreement management of some embodiments. The practice's billing 615 accesses the record of insurance benefits 610 as provided by the insurance 605 of the patient 600. In some embodiments, this is used to determine the proper structure for establishing the referral. In some embodiments, if multiple options are proper a determination is made on one option, such as to minimize paperwork or hassle, minimize patient out-of-pocket expenses, maximize reimbursement, or conform to the clinician agreement terms. Reconciliation block 625 operates with a referral network 620, which accesses a database of clinician agreements 635. In some embodiments the clinician agreements comprise rules and availability for procedures and services. In some embodiments the clinician provides prices for the services: in some embodiments this is provided in real time by the clinician's system as a spot price; in some embodiments price sheets 630 are provided ahead of time and can be located in the practitioner's system or elsewhere. In some embodiments, the practitioner's and clinician's billing systems negotiate or choose a price from a range of prices. A clinician billing system 640 can access the prices offered or confirmed for the specific service being provided at that point in time. A clinician point of sale 645 is then able to request the appropriate copay or per-use fee from the patient—if any. Some embodiments use the clinician agreement to determine that fee and/or the gathering workflow, in whole or part. Some embodiments use the reconciliation to determine the fee and/or the gathering workflow, in whole or in part. In some embodiments, the reconciliation block then confirms the final price and produces a reconciliation record to show the expected and final distribution of payments. In some embodiments, reconciliation happens in real time. In some embodiments, reconciliation happens after a settling period. In some embodiments, reconciliation results in an immediate transfer of funds or offsetting of accounts. In some embodiments, reconciliation results in a record that will be handled during a later sweep of accounts.

Note that, with the structure disclosed herein, the provider (or the patient if so desired) can have access to transparent and upfront pricing of the service. No common referral system today allows for referrals to be chosen based on pricing or compatibility: today this is usually a manual evaluation that, of all things, the patient is expected to perform. Furthermore, notice how the system can easily be configured to take advantage of variable pricing models, such as volume discounts or time of use discounts.

Also notice that the embodiments disclosed allow for a practitioner to increase their range or coverage of services by enlisting local resources close to the patient to leverage their own practice, which may be far from the patient. That being said, this invention may also be applied to when the practitioner is remote only temporarily, such as on vacation or visiting other practice sites and yet still trying to service her patients.

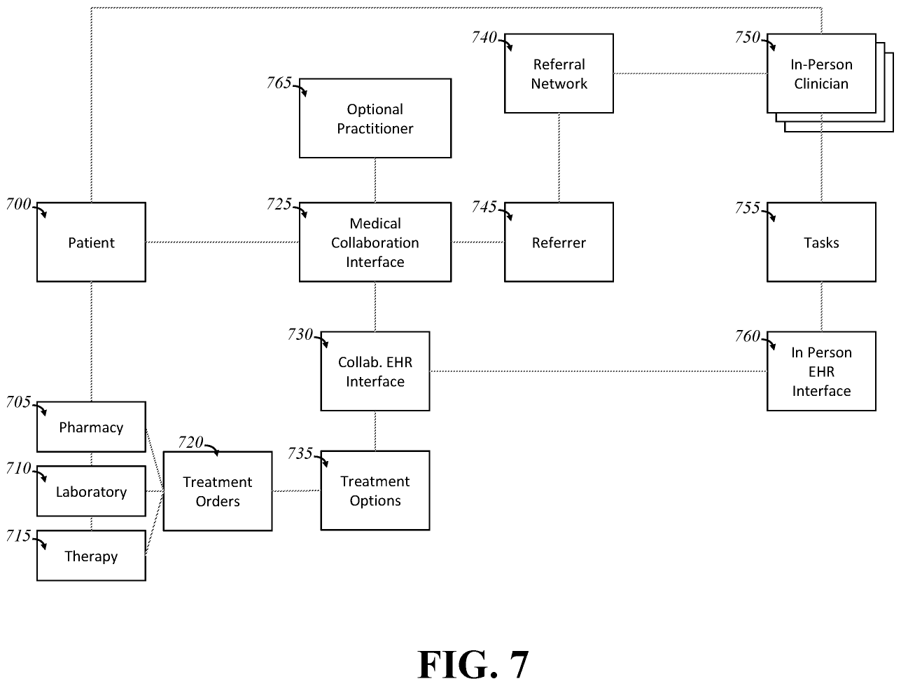

This invention can also be integrated into patient-directed care models. FIG. 7 shows some embodiments of patient-directed care, where a patient 700 interfaces with a medical collaboration interface 725 (such as defined in application Ser. No. 16/826,227 filed on Mar. 21, 2020 by the present inventor and hereby incorporated by reference). In some embodiments, the medical collaboration interface 725 is replaced with direct access to the EHR such as via 730, and/or treatment options 735 and treatment orders 720 (accessed directly or through the EHR). The practitioner 765 may connect to the medical collaboration interface 725. When the patient (or other users of the medical collaboration interface or its replacements) wishes to or has need for in person evaluation, then a referrer 745 is accessed as previously described to find local available resources to perform the evaluation. The embodiments that elaborate on the structure of FIG. 1 above can clearly be applied in this manner.

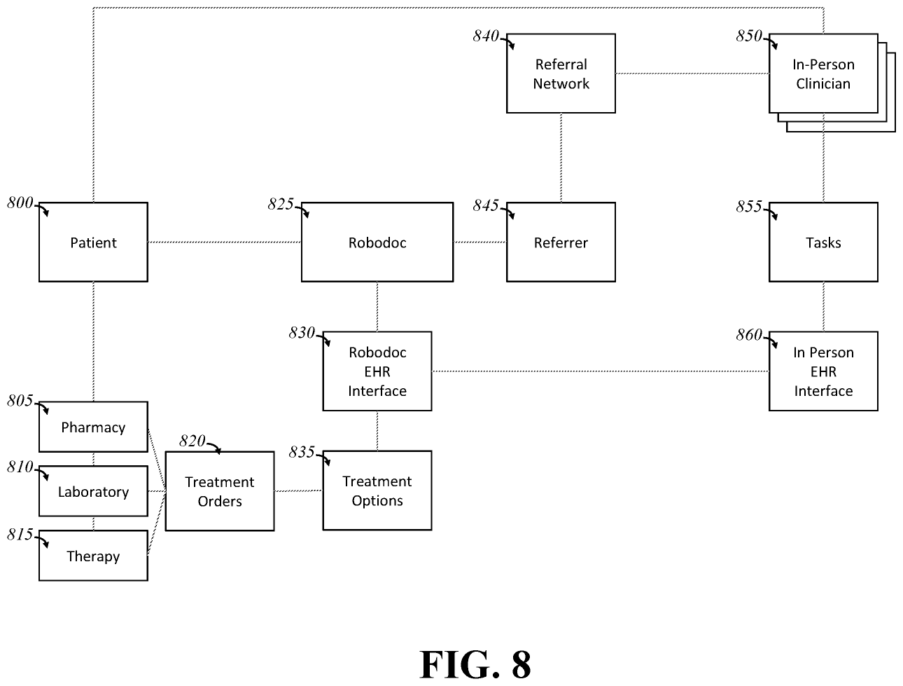

FIG. 8 shows some embodiments for robodocs. A robodoc is an automated, non-human platform or tool for performing partial or complete medical diagnostics, evaluations, or treatments. Some robodocs are designed to operate using natural human interfaces, such as text (chat) interfaces where they respond to questions and provide answers. Some work through the EHR system and generate reports based on the contents. Some work in other ways. What the robodocs have in common is that they are not corporeal, and so they cannot touch the patient, perform procedures on the patient, or actually deliver treatments. In some embodiments, the robodoc is remote, and the patient is not in a clinic at the time of seeking treatment. In some embodiments, the patient is at a clinic, but is interfacing with a robodoc. When a robodoc 825 decides that a corporeal task 855 needs to be performed, it accesses a referrer 845 to find through a referral network 840 an available and competent clinician 850, as previously disclosed. In the case where the patient 800 is already at a clinic, in some further embodiments the network is restricted to available staff on hand to perform the procedure. For example, if the patient presents to the robodoc with suspected tinea infection of the arm, and the robodoc decides through its processes that the suspected tinea should be confirmed by palpating the infection to feel for a raised border, it can request through the referrer that a medical assistant be summoned to attend to the patient to perform the palpation and enter the resulting impressions into the in person EHR interface 860 so that the robodoc can continue its course of diagnosis and treatment. In this example, the entire loop can be closed in real time, in which case the medical assistant who is summoned may be drawn from a pool of waiting or available assistants and the patient can receive a treatment order during the same encounter. In another example, the patient presents with a sore throat and indications point to a Streptococcus infection. The robodoc decides that the patient's throat needs to be swabbed and sent off to a laboratory. The robodoc accesses the referrer to reach out for a technician or nurse who can perform the swab and send the results off to the lab for immediate testing. In this case, the impressions are not necessary, and the results will come through the lab order. These examples can be applied to the case of where the patient is not in a clinic already. In the tinea example, the patient might be sent and scheduled to attend a local clinic to walk in and get a quick evaluation, and then asked to resume with the robodoc at the next convenient or appropriate time. In the Strep example, the patient might be directed to a clinician who has on-site or couriered laboratory services and the necessary swab, so that the swab can be handed off and the results made available as a result of the referral encounter. As with patient-direct medicine, the application of the further embodiments as shown in the figures and explained in the specification is straightforward to the robodoc model.

Moreover, the embodiments disclosed herein address another problem of robodocs. Robodocs, in most jurisdictions, are not recognized medical providers. They are treated as a tool only. A licensed human must be responsible for the encounter. However, nothing states what the balance must be between the human responsible for the encounter and the robodoc: the robodoc can be treated as a medical assistant for the purposes of the encounter, for example, so long as a licensed entity takes responsibility for the service. This is no different than with human physicians, where the medical assistant may handle most of the procedures, if not all, under the supervision of a licensed physician. A robodoc which can use the referral system to refer the patient to a general, nonspecific in person review from a licensed clinician of the robodoc's work will allow the robodoc to perform the procedures. Therefore, in some embodiments, the robodoc orders a referral (for at the same time or at another time) to a licensed clinician to ensure that a licensed clinician has participated in the encounter. In some embodiments, the referral is a general review, requesting that the clinician confirm the robodoc's diagnoses and orders. In some embodiments, the robodoc produces suggested orders, which are communicated to the clinician (such as via the EHR, the referrer, or an out of band method) to enter and make valid into the EHR, so as to cause the orders to take effect. In some embodiments, the referral is a second opinion referral. In some of these embodiments, the methods of split reimbursement as described prior allows for the payments to be appropriately divided between the owner/operator of the robodoc and the clinician signing off on the work of the robodoc. In some embodiments, this division is based on time spent, such as proration of the encounter fee. In some embodiments, the clinician that the patient is sent to is determined, at least in part, based on the fee structure of the clinician and the availability and willingness of the clinician to sign off on the work of a robodoc or this robodoc. This referral mechanism provides a strong way to ensure that a robodoc can legally treat patients in most jurisdictions.

This disclosure requires familiarity with the state of the art in medical diagnosis and treatment of patients. Terms like “detect” and “infer” are not necessarily absolutes, but may also refer to the increase in a determined value (such as likelihood or probability) or an increase in its confidence. Medical facts, statistical examples, numbers, and the like are for the purposes only of explaining the invention and its operation, and are merely illustrative.

It is the intent in this disclosure to teach not only the pure technological methods but the specific applications to various diseases and conditions.

Throughout this disclosure, multiple specific embodiments are listed that may be extensions of more general embodiments. It is to be understood that the combinations and subprocesses of these embodiments are also taught by this disclosure, as the combinations and subprocesses are able to be anticipated by those skilled in the art upon and only upon reading this disclosure. Furthermore, uses of the plural or the singular do not restrict the number of the item being mentioned: unless explicitly called out as not being so or being logically inconsistent, mentions of singular items are to be construed to also be plural and vice versa.

In the description herein, one or more embodiments of the invention are described, with process steps and functional interactions. Those skilled in the art would realize, after perusal of this application, that embodiments of the invention might be implemented using a variety of other techniques not specifically described, without undue experimentation or further invention, and that such other techniques would be within the scope and spirit of the invention. The use of the words “can” or “may” in regards to the structure and operation of embodiments is to be construed as referring to further embodiments and configuration options, and does not require further experimentation or invention.

The scope and spirit of the invention is not limited to specific examples disclosed therein, but is intended to include the most general concepts embodied by these and other terms.

Although the invention has been described with reference to several exemplary embodiments, it is understood that such descriptions and illustrations are not limiting. Changes may be made within the purview of the appended claims, as presently stated, without departing from the scope and spirit of the invention in its aspects. Although the invention has been described with reference to particular means, materials, machines, and embodiments, the invention is not intended to be limited to the particulars disclosed; rather, the invention extends to all functionally equivalent structures, methods, machines, and uses such as are within the scope of the invention and claims.

This disclosure lists sufficient details to enable those skilled in the art to construct a system around or using the novel methods of the contained inventions, without further discovery or invention.

I claim:

1. A computer-implemented method for managing and storing electronic health records data for integrated remote and in-person encounters comprising:
a) providing a remote medical encounter for a patient;
b) receiving a request to assign at least one procedure code to at least part of the remote medical encounter at a central electronic health records system that contains medical procedures data;
c) identifying from the medical procedures data and the request to assign at least one procedure code that at least one of the procedure codes requested to be assigned requires at least one in-person task;
d) identifying an in-person provider capable of performing at least part of the in-person task;
e) requesting over a network to an electronic health records system of the in-person provider an electronic order for the at least part of the procedure that must be performed in person;
f) retrieving, over a network from the in-person provider patient medical record data concerning the in-person task;
g) integrating within the central electronic records system the retrieved patient medical data record into an aggregated procedure record; and
h) assigning the at least one procedure code to the at least part of the remote medical encounter that is associated with the aggregated procedure record within the central electronic health records system.

2. The method in claim 1 wherein the request to assign at least one procedure code is derived at least in part from an action by at least one of: a human practitioner, an automated electronic diagnostics engine, a patient-directed medical system, and a robodoc.

3. The method in claim 1 wherein said performing of the in-person task is electronically scheduled on behalf of the patient.

4. The method in claim 1 wherein a referral network is used to identify the in-person provider, via at least one of: directly, and through a clinic to which said in-person provider is affiliated with.

5. The method in claim 4 wherein said identification of said in-person provider is based at least in part on at least one of:
ability for billing to be reconciled, and ability for the patient to see expected costs of performance of the in-person task.

6. The method in claim 1 wherein the request of an electronic order and the retrieval of the medical record data concerning the in-person task occur over an EHR-to-EHR bridge.

7. The method in claim 1 performing automated reconciliation billing for the at least one procedure code comprising further steps of:
submitting an electronic reimbursement request for the at least one procedure code using one provider identifier based on the aggregate procedure record;
creating a reconciliation billing entry for the remote provider showing amounts owed to the in-person provider.

8. An electronic health records system, comprising:
a) a medical database system storing patient medical data records and medical procedures data;
b) a server coupled to the medical database system, the server comprising at least one processor coupled to a memory and configured to:
i. receive a request to assign at least one procedure code to at least part of a remote medical encounter with a patient;
ii. identify from the medical procedures data and the request to assign at least one procedure code that at least one of the procedure codes requested to be assigned requires at least one in-person task;
iii. identify an in-person provider capable of performing at least part of the in-person task;
iv. request over a network to an electronic health records system of the in-person provider an electronic order for the at least part of the procedure that must be performed in person;
v. retrieve, over a network from the in-person provider patient medical record data concerning the in-person task;
vi. integrate the retrieved patient medical data record into an aggregated procedure record; and
vii. assign the at least one procedure code to the at least part of the remote medical encounter that is associated with the aggregated procedure record.

9. The system in claim 8 wherein the request to assign at least one procedure code is derived at least in part from an action by at least one of: a human practitioner, an automated electronic diagnostics engine, a patient-directed medical system, and a robodoc.

10. The system in claim 8 wherein said performing of the in-person task is electronically scheduled on behalf of the patient.

11. The system in claim 8 wherein a referral network is used to identify the in-person provider, via at least one of: directly, and through a clinic to which said in-person provider is affiliated with.

12. The system in claim 11 wherein said identification of said in-person provider is based at least in part on at least one of: ability for billing to be reconciled, and ability for the patient to see expected costs of performance of the in-person task.

13. The system in claim 8 wherein the request of an electronic order and the retrieval of the medical record data concerning the in-person task occur over an EHR-to-EHR bridge.

14. The system in claim 8, the at least one processor further configured to performing automated reconciliation billing for the at least one procedure code.
